(19) United States
(12) Patent Application Publication
(10) Pub. No.: US 2003/0198646 A1
(43) Pub. Date: Oct. 23, 2003

(54) Compositions and methods for inducing new hair follicle formation and hair growth in a desired orientation

(76) Inventor: Kurt Stricker Stenn, Princeton, NJ (US)

Correspondence Address: MICHAEL BEST & FRIEDRICH, LLP, ONE SOUTH PINCKNEY STREET, P O BOX 1806, MADISON, WI, 53701

(21) Appl. No.: 10/123,984

(22) Filed: Apr. 17, 2002

(51) Int. Cl. A61K 39/00

(52) U.S. Cl. 424/198.1; 424/93.7

(57) ABSTRACT

The present invention relates to compositions and methods for inducing hair follicle formation and subsequent hair growth in a mammal in a cosmetically acceptable orientation. The method further comprises implanting papilla cells or growth factors under or in contact with the epidermal layer with a vehicle to direct growth of the epidermis and thus the formation of nascent hair follicles. The vehicle is removed after hair follicle formation has begun. The present invention further comprises an apparatus for the packaging and delivery of papilla cells.

### BACKGROUND OF THE INVENTION

Hair follicles are unique to mammalian skin. The hair follicle is a down-growth of the primitive epidermis, extending into the deeper layers of the skin. At the base of the hair follicle resides a plug of cells known as the follicular or dermal papilla (Stenn and Paus, 2002, Physiol. Rev., 81:449). The papilla is necessary to the normal cycling of the hair follicle, (Oliver, 1966, Embryol. Exp. Morph. 15:331; Oliver, Embryol. Exp. Morph. 16:231) and thus growth of the hair shaft. The hair shaft is a thread shaped structure made of tightly coherent epithelial cells filled with keratin filaments and filament aggregating proteins.

Hair loss is caused by a number of factors. In human male pattern baldness, hair follicles on the front and top of the scalp are susceptible to androgens and undergo, in susceptible individuals, the transformation from a large to a microscopically small follicle, appearing clinically as hair loss. It is estimated that 20% of women will also experience some sort of hair loss in their lifetimes often characterized by a thinning of hair on the top of the scalp. In aging there is a diffuse loss of hair. Further, different disease states, such as the scarring conditions, e.g. associated with the cicatrial alopecias, thermal bums or pressure injuries can result in significant hair loss. The end result of hair loss, regardless of cause, can have significant psychological, social and sexual consequences with a loss of self-esteem and personal confidence.

The treatment and solutions to hair loss have varied considerably over time. Wigs, toupees, and hair extensions can conceal balding areas but do not generate new growth. Two available medications (minoxidil and finasteride) may slow further hair loss but none are available which actually lead to the regeneration of new hair follicles. The current and widely used approach to replace thinning or missing hair follicles is hair transplant surgery.

For over thirty years, hair transplant surgery has been the only viable method to replace active hair follicles to a bald area. In this procedure hair follicles are surgically removed from an area of the scalp which does not undergo balding (occipital area) and transplanted to bald areas (frontal and crown areas). The hair grafts range from 1-2 follicles to 10-15 follicles per graft. There are limitations to this procedure. First, this transplant surgery is very slow and labor intensive. Since it is not uncommon to transplant between 750 and 1500 follicles in one transplant session, the procedure generally requires one complete working day. This procedure is slow and intensive because each donor follicle must be dissected from the donor site before it is implanted. Second, multiple operative sessions may be needed to satisfy a cosmetic effect. Finally, and most importantly, follicles from a donor site must be sacrificed for the receptor site. This donor site then potentially scars but most certainly is left with fewer follicles. This fact eliminates the procedure as an option for those without an appropriate donor area. In order to minimize the sacrifice of follicles, other means of inducing new follicle formation are needed. There is also a need for a system which generates new follicles without sacrificing original follicles which implants large number of follicles quickly and cheaply with an optimal cosmetic result.

To this end, since dermal papilla cells alone can induce new hair follicles and thus hair shaft formation when implanted under the epidermal layer, implantation of inductive papilla cells as a basis for a new therapy would appear the most promising approach. Papilla cells alone can induce epidermal cells to form new viable hair follicles (Cohen, 1961, Embryol. Exp. Morph., 9:117, 1961; Oliver, 1967, Embryol. Exp. Morph., 18:43; 1970, Embryol. Exp. Morph., 23:219; Oliver et al., U.S. Pat. No. 4,919,664; Jahoda, 1992, Development, 115:1103; Reynolds et al., 1991, Ann. N.Y. Acad. Sci., 642:226; Reynolds et al., 1999, Nature 402:33). Not only are autologous papilla grafts effective in inducing new follicles but it has been shown that papilla cells are immune privileged so that allogeneic transplants are also effective (Reynolds et al., 1999, Nature 402:33). Finally, inductive papilla cells can be maintained and propagated in cell culture, (Cooley et al., PCT/US98/13754; Kishimoto et al., 2000, Genes. Dev. 14:1181) allowing for the accumulation of many cells available for implantation, reducing the need for abundant donor tissue, reducing the number of repeat procedures and minimizing the cannibalization, or sacrifice, of healthy follicles from the donor site as is experienced in conventional hair transplant surgery.

One of the problems of papilla transplant surgery is that the efficiency of new follicle formation is low and the orientation of the resultant follicles is unpredictable. Recent progress in elucidating the identity of several growth and transcription factors involved in hair follicle cycling and hair shaft orientation suggest that their use may enhance the efficiency of surgery and the cosmetic result. TGFα and its receptor (Nixon et al., 1996, J. Histochem. Cytochem, 44:377) Krox-2, TGFβRII (Gambardella et al., 2000, Mech. Dev. 96:215), sonic hedgehog (Chiang et al., 1999, Dev. Biol. 205:1), homeobox protein (Widelitz et al., 1997, Microsc. Res. Tech., 15:38), and Notch proteins (Lin et al., 2000, Development 127:2421) have all been observed to exhibit eccentric expression in the hair follicle, and may therefore play a role in new follicle induction, patterning, survival and orientation.

Previous disclosures describing the introduction of papilla cells into mammalian skin without an accompanying structure do not describe a method to orient the direction or growth of the follicle and resulting hair shaft, therefore possibly producing a shaft growing in an unpredictable and cosmetically unacceptable direction.

Previous disclosures describing methods to implant papilla cells in conjunction with a rigid shaft to direct the growth and orientation of the hair follicle have also failed to realize the full potential of papilla cell implantation. Cooley et al. (PCT/US98/13754), describes the co-introduction of papilla cells with a rigid shaft composed of an absorbable suture material, followed by covering the wound with an occlusive dressing, or keeping the wound open. A shaft made of an absorbable material may dissolve at an unpredictable rate, possibly before formation of a nascent hair follicle. Further, the use of a biologically active compound, such as an absorbable suture material, may provoke an inflammatory immune response at the site of implantation. It has been observed that any inflammatory or foreign body reaction to a biologically active material will impede the formation of new follicles so that any biodegradable material which elicits a foreign body reaction in the vicinity of a regenerating follicle would be counterproductive—ineffective at least and pathological at worst.

There is a long felt need to develop a less destructive and less expensive method for replacing hair growth, a method which induces new hair follicles with a predictable success rate and cosmetic result. The present invention meets this need.

### BRIEF SUMMARY OF THE INVENTION

The present invention comprises a method of inducing hair growth in a desired orientation wherein the method comprises contacting an epidermal layer of the skin of a mammal with a vehicle comprising a stiff shaft and at least one papilla cell. The method further comprises securing the vehicle to the epidermal layer in a desired orientation and then removing the vehicle from the epidermal layer after a period of time, thereby inducing hair growth in the desired orientation.

In another aspect of the present invention, the mammal is a human.

In yet another aspect of the present invention, the stiff shaft is a tube, a rigid thread, or a rod.

In still another aspect of the present invention, the papilla cells are autologous or allogeneic.

In one aspect of the present invention, the papilla cells are propagated in cell culture.

In another aspect of the present invention, the papilla cells are obtained from a human.

In still another aspect of the present invention the vehicle is removed after a period of time from about two days to about ten days.

In one aspect of the present invention, the vehicle comprises a growth factor.

In another aspect of the present invention, the vehicle comprises an eccentrically expressed growth/transcription factor wherein the growth/transcription factor may be a member of the Wnt, sonic hedgehog, Krox-20, homeobox, Notch, transforming growth factor-alpha and insulin-like growth factor families.

The present invention also comprises a method for inducing hair growth in a desired orientation in a mammal, wherein the method comprises contacting an epidermal layer of the skin with a vehicle comprising a stiff shaft wherein the vehicle comprises a growth of growth/transcription factor. The method further comprises securing the vehicle to the epidermal layer in a desired orientation and then removing the vehicle from the epidermal layer after a period of time, thereby inducing hair growth in the desired orientation.

In another aspect of the present invention, the mammal is a human.

In yet another aspect of the present invention, the stiff shaft is a tube, a rigid thread, or a rod.

In still another aspect of the present invention the vehicle is removed after a period of time from about two days to about ten days.

In one aspect of the present invention, the vehicle comprises a growth factor.

In another aspect of the present invention, the vehicle comprises an eccentrically expressed growth/transcription factor wherein the growth/transcription factor may be a member of the Wnt, sonic hedgehog, Krox-20, homeobox, Notch, transforming growth factor-alpha and insulin-like growth factor families.

The present invention also includes an apparatus for the packaging and placement of papilla cells wherein a tube comprising papilla cells is sealed to form individual segments of the tube, thereby forming an apparatus for the packaging and placement of papilla cells.

In one aspect of the present invention, one or more tubes are interconnected by a flexible device.

In another aspect of the present invention, the tube comprises a biologically inert plastic.

In yet another aspect of the present invention, the tube is sealed into segments by heat sealing, chemical sealing, or mechanical means.

In still another aspect of the present invention, the segments are detached to form a vehicle comprising a stiff shaft and at least one papilla cell.

### BRIEF SUMMARY OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1D, depicts the method of implanting a vehicle and papilla cells into the epidermal layer of a mammal to generate a new hair follicle in a cosmetically acceptable orientation.

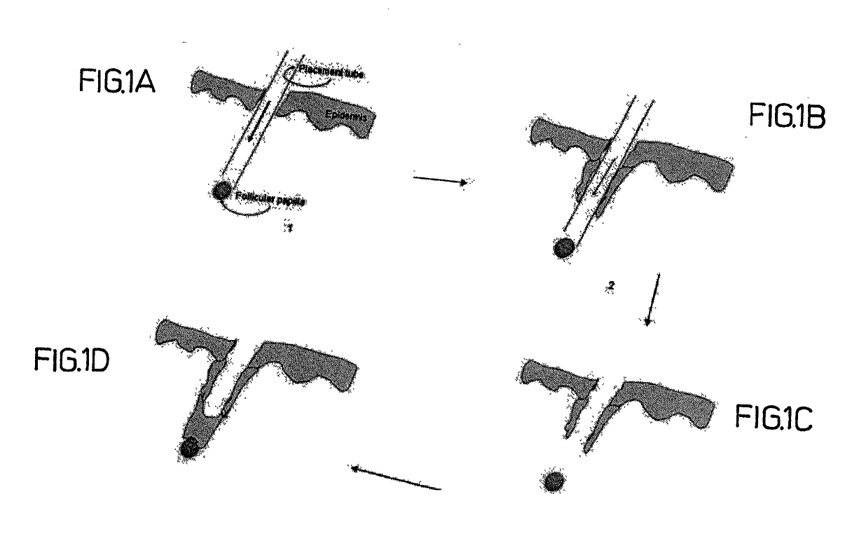

FIG. 2 depicts a device to facilitate the packaging, insertion, and placement of vehicles and papilla cells. The device comprises a plurality of long tubes interconnected by a flexible device and segmented into sections that when detached from the long tube form vehicles for implantation.

FIG. 3 depicts a method of packaging papilla cells in a long tube.

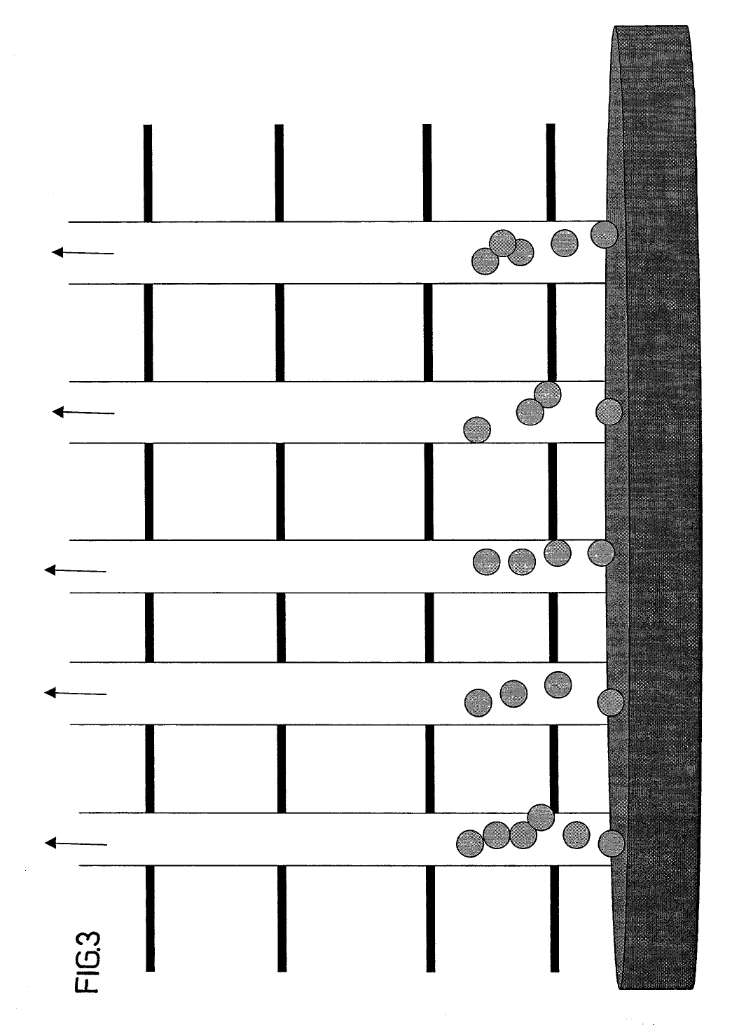

FIG. 4 depicts early hair follicle formation after the implantation of papilla cells.

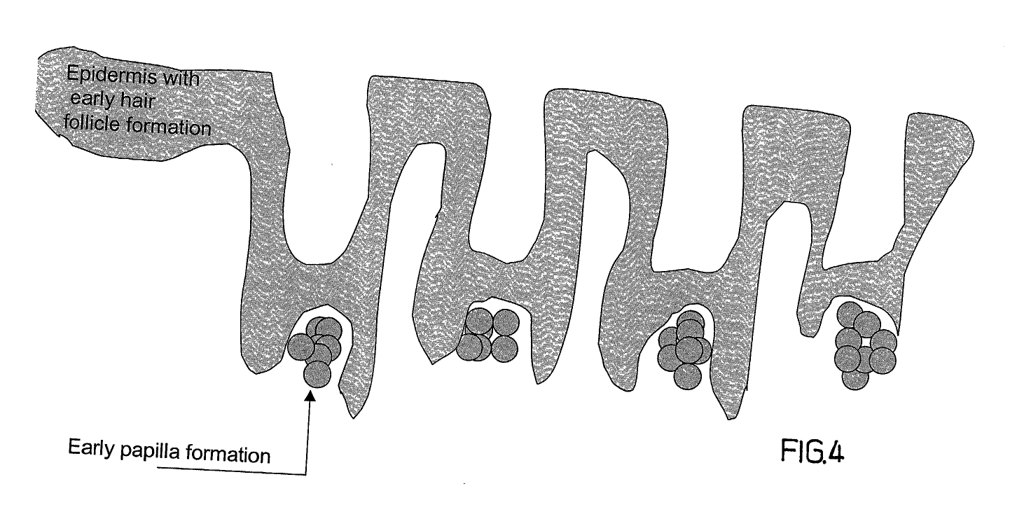

### DETAILED DESCRIPTION OF THE INVENTION

The invention includes a novel method for delivering hair inductive papilla cells or growth factors into the superficial skin of a human, resulting in new follicle formation with high success rate of follicle growth and a cosmetically acceptable orientation. This method overcomes the destructive or cannibalization aspect of current-day hair transplant surgery since minimal donor tissue is needed.

The present invention is based in part on the discovery that human follicular papilla cells can be implanted in or subjacent to the epidermal tissue, resulting in hair follicle formation and subsequent hair growth. According to the present invention, papilla cells are introduced into or in contact with human skin in conjunction with a removable, biologically inert vehicle capable of inserting papilla cells under or in contact with the epidermal layer of the skin. The inert vehicle carries the papilla cells into the dermis and stimulates the downgrowth of the epidermal cells along the vehicle toward the inserted papilla cells. It is known that inductive papilla cells attract epidermal cells toward them (Arase et al., 2001, J. Dermatol. 17:667; Fuji et al., 2001, J. Dermatol. Sci. 25:206). The vehicle is inserted into or in contact with the skin through the epithelial layer in an orientation found to be cosmetically acceptable. The papilla cells and vehicle are secured to the skin and left in the insertion site to facilitate the formation of an epithelial cell column that grows down and around the insert and will eventually form the hair follicle. The vehicle may be treated or coated with a growth factor or a transcription factor, or a construct expressing these factors, to facilitate hair follicle formation and hair growth. The vehicle is removed from the epidermal layer after a prescribed period of time.

The present invention also relates in part to the discovery that the formation or regeneration of a hair follicle can be induced by contacting the epidermal tissue with certain growth factors in the absence of added papilla cells. According to the present invention, a removable, biologically inert vehicle capable of contacting the epidermal layer is inserted into the skin. The vehicle is inserted into the epithelial layer in an orientation found to be cosmetically acceptable and secured to the skin and left in the insertion site to facilitate the formation of an epithelial cell column that will eventually form the hair follicle. The vehicle may be treated or coated with a growth factor or a transcription factor, or a construct expressing these factors, to facilitate hair follicle formation and hair growth. The vehicle is removed from the epidermal layer after a prescribed period of time.

The present invention also includes a novel device to facilitate the packaging, insertion, and placement of vehicles in the epithelial layer of skin with or without papilla cells. According to the present invention, a vehicle, which may or may not comprise papilla cells and may or may not comprise a growth or transcription factor, but does comprise at least one of these components, is attached to other vehicles through an interconnecting device. The vehicles may be longer than what is needed to be inserted into the skin, and therefore may be segmented to create shorter individual vehicles out of one or more long vehicles. The segmented vehicles may be separated before insertion by simply cutting or snapping them apart.

The present invention thus relates to a method of inducing hair growth in a desired orientation in a mammal. The method is useful in regenerating hair growth in persons with alopecia, especially male and female pattern baldness, and those who have hair loss due to scarring (such as scar resulting from burns, trauma, radiation injury, chemotherapy, pressure, etc.). Although this method would be applied most often to scalp skin, it is applicable to hair growth on any skin surface over the complete body (e.g. eyelids, eyebrows, beard, inguinal area, etc.).

In one embodiment of the invention, papilla cells for implantation are derived from hair-growing scalp biopsies. In another embodiment, papilla cells for implantation are obtained from follicle-containing skin grafts. Methods of obtaining papilla cells from donor sources will be readily apparent to those of ordinary skill in the art (Jahoda and Oliver, 1981, Br. J. Dermatol., 105:623; Messenger, 1984, Br. J. Dermatol, 110:685; Williams et al., 1994, Br. J. Dermatol., 130:290).

In yet another embodiment, papilla cells for implantation are derived from a primary cell culture. In a preferred embodiment, papilla cells for implantation are derived from a propagated cell culture.

In one embodiment, the papilla cell culture is maintained in the presence of feeder cells and/or papilla cell growth factors. The conditions and requirements for the maintenance and propagation of inductive papilla cell cultures are well known to those of ordinary skill in the art (Kishimoto et al., 2000, Genes. Dev. 14:1181; Cooley et al., PCT/US98/13754).

In the present invention, the vehicle is removed from the skin some time after implantation. In one embodiment of the invention, the vehicle is made of a rigid material. In another embodiment, the vehicle is made from materials including but not limited to plastic, metal, or a stiff fibrous thread. The core material of the vehicle is physiologically and biologically inert. In another embodiment, the vehicle is a tube. In another embodiment, the vehicle is a rod. In a preferred embodiment, the vehicle is a biologically inert plastic tube. In one embodiment, the tube to be inserted is about 0.01 mm to about 3.0 mm in outside diameter and about 1 mm to about 20 cm in length. In one embodiment, the inside diameter of the tube is from about 0.01 mm to about 3.0 mm. In one embodiment, the plastic tube comprises, but is not limited to polypropylene, polyethylene, polystyrene, TEFLON, and polycarbonate. In still another embodiment, the vehicle is shaped to closely resemble the shape of existing or desired hair follicles. This includes, but is not limited to, modifying the cross-sectional profile of the vehicle to a desired shape, placing bends, curves, or coils in the vehicle, or altering the diameter of the vehicle.

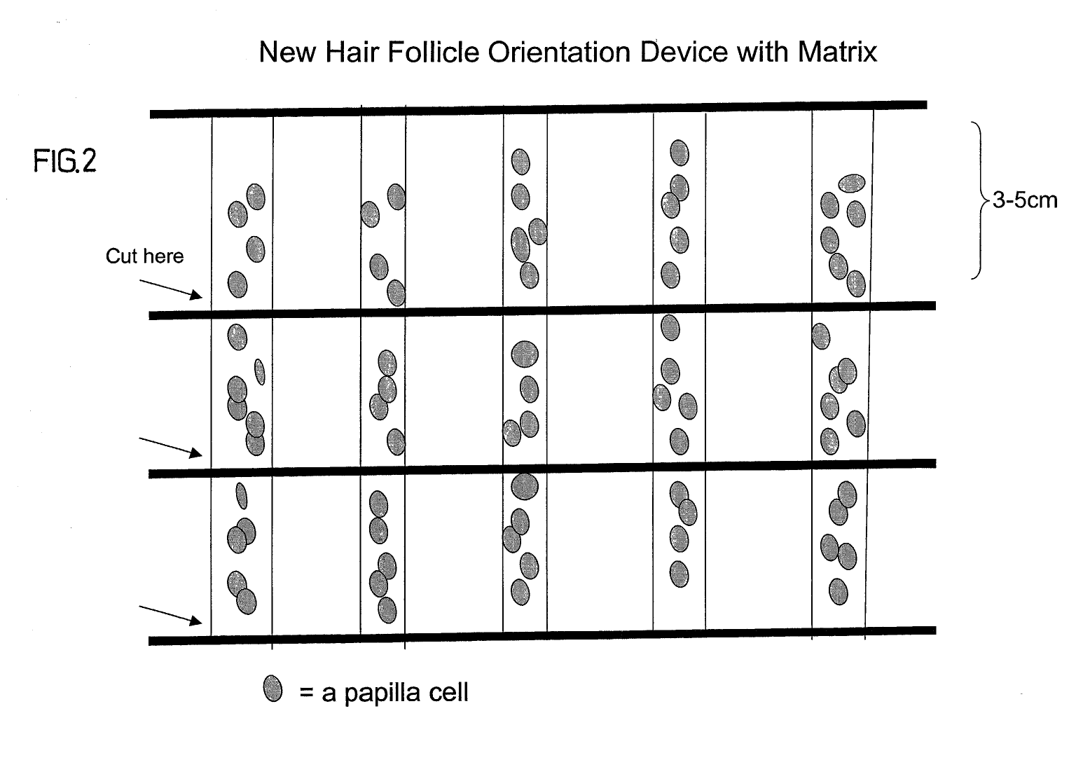

In one embodiment of the present invention, a tube is segmented and separated to form shorter tubes to serve as vehicles for insertion into the skin (FIG. 2). In a preferred embodiment of the present invention, the vehicle is a portion of a tube comprising papilla cells that is segmented into sealed compartments. In one embodiment of the present invention, the tube is sealed by methods including, but not limited to heat sealing, chemical sealing (e.g. epoxies and glues), and sealing by a mechanical means (e.g. a clamp, crimping, or wire tie).

In one embodiment of the present invention, the tubes are interconnected. In still another embodiment of the present invention, the tubes are interconnected by a flexible device to allow manipulation and arrangement of the tubes or vehicles in a desired orientation. In one embodiment of the present invention, the flexible device includes, but is not limited to a thread, a string, paper, a thin piece of metal, a piece of plastic, and an adhesive tape. In a preferred embodiment of the present invention, the flexible device interconnects from about 1 to about 1000 tubes. More preferably, the flexible device interconnects from about 2 to about 100 tubes. Even more preferably, the flexible device interconnects from about 3 to about 50 tubes. Still more preferably, the flexible device interconnects from about 3 to about 5 tubes.

In one embodiment of the present invention, the tubes are interconnected by a flexible device at a plurality of locations. In a preferred embodiment of the present invention, the flexible devices are attached to the tube about every 5 mm to about every 20 cm.

In a preferred embodiment, the papilla cells are contained within the tube prior to implantation. In one embodiment, papilla cells suspended in an aqueous medium are sealed in each segment of the tube prior to implantation. In another embodiment, clumps of papilla cells are sealed in each segment of the tube prior to implantation. In one embodiment of the present invention about 1 to about 10⁷ papilla cells are sealed in a segment of the tube. In a preferred embodiment, the individual sealed segments of the tube are detached to form the vehicle for insertion. In one embodiment of the present invention each segment is detached from the tube to form the vehicle by the use of a cutting instrument, mechanical means or heat prior to implantation.

In one embodiment of the present invention, the vehicle comprises growth or transcription factors as a protein or nucleic acid construct either with or without papilla cells. Methods of making a vehicle comprising growth or transcription factors are described herein.

In a preferred embodiment of the invention, the vehicle is placed in contact with the epidermal cells of the skin to facilitate the growth and “walling-off” of the vehicle by the downgrowing adjacent epidermis, as epidermis reacts to an inert foreign body by attempting to wall it off from the surrounding tissues (Clark et al., 1988, Mol. Cell. Biol. of Wound Repair, Plenum Pub., Co. New York). Such downgrowing cells contain populations which can respond to the inductive properties of the papilla (Ferraris et al., 1997, Int J Dev Biol, 41:491). In yet another preferred aspect of the invention, the vehicle is removed after the formation of a nascent hair follicle.

In one embodiment of the present invention, the vehicle is coated or otherwise comprises a growth factor which facilitates the attraction, motility, and subsequent growth of epidermal cells around the vehicle. The contemplated growth factors include, but are not limited to members of the following growth factor families; basic fibroblast growth factor, fibronectin, epidermal growth factor, noggin, transforming growth factor-beta, transforming growth factor-alpha, trefoil factors, platelet-derived growth factor, vascular endothelial growth factor, insulin-like growth factors, hepatocyte growth factor, fibrin, collagen, and laminin (Sigma Chemical Co. St. Louis, Mo.). The contemplated growth factor may be applied to the vehicle in a solution and allowed to dry, or applied to the vehicle as a dry solid, or may be incorporated into the material of the vehicle. Other methods of incorporating such a growth factor into the vehicle will be well known to those of ordinary skill in the art. Concentrations of the growth factor to be incorporated into the vehicle may be from about 0.01 ng to about 100 mg final dried weight of growth factor per vehicle.

In another embodiment, the growth factors may be incorporated into the vehicle as a DNA construct capable of expressing the non-limiting list of growth factors above. In one embodiment, the growth factor is incorporated into the vehicle as a vector or expression vector. In one embodiment, the vehicle comprises components for cell free transcription and translation. Such components may include an RNA polymerase and cell extracts from eukaryotic or prokaryotic sources. Such components are well known to those of ordinary skill in the art, and are available from many sources (Ambion, Austin Tex.).

Methods to make and use vectors and expression vectors are well known to those of ordinary skill in the art (Sambrook et al. 1989, Molecular Cloning: A Laboratory Manual, Cold Spring Harbor Laboratory, New York).

In another embodiment, the non-limiting list of growth factors are delivered with or incorporated into the vehicle as a viral vector. Methods to accomplish this are well known to those skilled in the art (Sato et al., 1999, J. Clin. Invest. 104:855).

In a preferred embodiment, the vehicle is coated or otherwise comprises an eccentrically expressed growth/transcription factor which plays a role in the growth and positioning of a newly formed hair follicle. Contemplated growth/transcription factors include, but are not limited to; Krox-20, TGF-betaRII (Gambardella et al., 2000, Mech. Dev., 96:215), sonic hedgehog (Chiang et al., 1999, Dev. Biol., 205:1), a homeobox protein (Widelitz et al., 1997, Microsc. Res. Tech., 38:452), a Wnt family protein (Kishimoto et al., 2000, Genes. Dev. 14:1181), transforming growth factor-alpha, insulin-like growth factor (Philpott et al., 1994, J. Invest. Derm., 102:857) and Notch proteins (Lin et al., 2000, Development, 127:241). The contemplated growth/transcription factor may be applied to the vehicle in a solution and allowed to dry, or be applied to the vehicle as a dry solid, or it may be incorporated into the material of the vehicle. Other methods of incorporating a growth/transcription factor into the vehicle will be well known to those of ordinary skill in the art. Concentrations of growth/transcription factors to be incorporated into the vehicle may be from about 0.01 ng to about 100 mg final dried weight of growth/transcription factor per vehicle.

In another embodiment, the non-limiting list of growth/transcription factors above may be incorporated into the vehicle as a DNA construct capable of expressing a protein. In one embodiment, the growth/transcription factor is incorporated into the vehicle as a vector. In one embodiment, the vehicle comprises components for cell free transcription and translation. Such components may include an RNA polymerase and cell extracts from eukaryotic or prokaryotic sources. Such components are well known to those of ordinary skill in the art, and are available from many sources (Ambion, Austin Tex.).

In another embodiment, the non-limiting list of growth/transcription factors are delivered with or incorporated into the vehicle as a viral vector. Methods to accomplish this are well known to those skilled in the art (Sato et al., 1999, J. Clin. Invest., 104:855).

In one embodiment of the present invention, the area of the skin in which the vehicle and papilla cells is to be inserted may be treated with physical and pharmacological methods to facilitate the formation of hair follicles and hair growth. Such treatments include but are not limited to ultrasound, ultraviolet irradiation, massage, and the application of topical compositions such as minoxidil (Sigma Chemical Co. St. Louis, Mo.). Other methods to facilitate the formation of hair follicles will be well known to those of ordinary skill in the art.

In a preferred embodiment of the invention, the vehicle and papilla cells are inserted into the skin in order to contact the native epidermal cells with the papilla cells, and to achieve a cosmetically acceptable placement of the hair follicle. The depth, location, and insertion angle of the vehicle and papilla cells will be readily apparent to those of ordinary skill in the art. The vehicle is then removed after a prescribed period of time, from about 2 days to about 10 days after implantation. The vehicle may be removed at an earlier time or at a later time, depending on the rate of formation of a nascent hair follicle. The time between implantation and removal will be apparent to those of ordinary skill in the art.

The vehicle and papilla cells may be inserted in any way to achieve cosmetically acceptable hair follicle placement and orientation. In a preferred embodiment of the current invention, non-naturally occurring orifices are made in the area of skin in which the vehicle and papilla cells are implanted. In another embodiment of the present invention, single non-naturally occurring orifices are made in the skin one at a time, and the vehicle and papilla cells are implanted singly. In yet another embodiment of the present invention, multiple non-naturally occurring orifices are made at one time, and the vehicle and papilla cells are implanted singly. In a preferred embodiment of the present invention, multiple non-naturally occurring orifices are made at one time, and the vehicle and papilla cells are implanted in groups. In still another embodiment of the invention a plurality of vehicles and papilla cells are placed in a support, and implanted simultaneously. The vehicle may be implanted using any device which facilitates the implantation of multiple vehicles into the skin.

In one embodiment of the invention, the vehicle and papilla cells are implanted in one procedure at one time. In another embodiment of the invention, the vehicle and papilla cells are implanted in a number of procedures at different times.

In a preferred embodiment of the invention, the vehicle is secured to the skin to allow the migration and growth of epidermal cells adjacent to the vehicle. In one embodiment, the vehicle is secured by its placement in the skin. In still another embodiment, the vehicle is secured by a medical adhesive, including but not limited to DERMABOND™ or LIQUIDERM™ (Closure Medical Corp. Raleigh, N.C.). In another embodiment, the vehicle is held in place by sutures, staples, or adhesive tape. In the present invention, the vehicle is left in place for a prescribed period of time and is then removed from the skin. The length of time necessary to allow formation of a nascent hair follicle comprising epidermal cells around the vehicle will be readily apparent to those of ordinary skill in the art.

Definitions

The articles “a” and “an” are used herein to refer to one or to more than one (i.e. to at least one) of the grammatical object of the article. By way of example, “an element” means one element or more than one element.

The term “plurality” is used herein to refer to two or more.

The term “papilla cell” is used herein to refer to a cell type known as a dermal papilla cell or a follicular papilla cell, or by any other name meant to construe a group of cells derived from the base of a hair follicle having hair follicle inductive properties.

The terms “cosmetically acceptable” is used herein to refer to the placement of papilla cell implants so that resulting hair follicles will be oriented in a predictable direction as determined by the practitioner performing the implant procedure.

The term “desired orientation” is used herein to refer to the placement of papilla cell implants so that resulting hair follicles will be oriented in a predictable direction as determined by the practitioner performing the implant procedure.

The term “autologous” is used herein to refer to a transplant of tissue from one individual to the same individual.

The term “allogeneic” is used herein to refer to a transplant of tissue from one individual to a different individual of the same species.

The term “growth factor” is used herein to refer to a biological growth factor comprising a peptide or protein, that when contacting epidermal cells, facilitates their proliferation, growth, patterning, orientation or motility.

The term “growth/transcription factor” is used herein to refer to a biological growth factor comprising a peptide or protein, that when contacting epidermal cells, facilitates their proliferation, growth, patterning, orientation or motility.

The term “eccentrically expressed” is used herein to refer to the presence of a molecule in a spatially and temporally irregular pattern.

The term “epidermal layer” is used herein to refer to the layer of tissue that overlies the dermis, forming the outer integument of the body.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and nonviral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “non-naturally-occurring” orifice of an animal is an orifice (e.g. an incision, puncture, wound, etc.) which is not normally present in an animal which is not afflicted with a disease or disorder.

The term “biologically inert” is used herein to refer to a substance that while in contact with an biological entity, does not elicit a reaction in that entity, nor does the entity elicit a reaction in that substance.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of inducing hair growth in a desired orientation in a mammal, the method comprising contacting an epidermal layer of the skin of the mammal with a vehicle comprising a stiff shaft and at least one papilla cell, securing the vehicle to the epidermal layer in the desired orientation, and removing the vehicle from the epidermal layer after a period of time, thereby inducing hair growth in the desired orientation.

2. The method of claim 1, wherein the mammal is a human.

3. The method of claim 1, wherein the stiff shaft is a tube.

4. The method of claim 1, wherein the stiff shaft is a rigid thread.

5. The method of claim 1, wherein the stiff shaft is a rod.

6. The method of claim 1, wherein the papilla cells are autologous.

7. The method of claim 1, wherein the papilla cells are allogeneic.

8. The method of claim 1, wherein the papilla cells are propagated in cell culture.

9. The method of claim 1, wherein the papilla cells are obtained from a human.

10. The method of claim 1, wherein the vehicle further comprises a growth factor.

11. The method of claim 1, wherein the period of time is between about two and about ten days.

12. The method of claim 1, wherein the vehicle further comprises an eccentrically expressed growth/transcription factor.

13. The method of claim 12, wherein the eccentrically expressed growth/transcription factor is member of the Wnt growth/transcription factor family.

14. The method of claim 12, wherein the eccentrically expressed growth/transcription factor is member of the sonic hedgehog growth/transcription factor family.

15. The method of claim 12, wherein the eccentrically expressed growth/transcription factor is a member of the Krox-20 growth/transcription factor family.

16. The method of claim 12, wherein the eccentrically expressed growth/transcription factor is a member of the homeobox growth/transcription factor family.

17. The method of claim 12, wherein the eccentrically expressed growth/transcription factor is a member of the Notch growth/transcription factor family.

18. The method of claim 12, wherein the eccentrically expressed growth/transcription factor is a member of the transforming growth factor-alpha growth/transcription factor family.

19. The method of claim 12, wherein the eccentrically expressed growth/transcription factor is a member of the insulin-like growth factor growth/transcription factor family.

20. A method of inducing hair growth in a desired orientation in a mammal, the method comprising contacting an epidermal layer of the skin of the mammal with a vehicle comprising a stiff shaft, wherein the vehicle comprises a growth factor or growth/transcription factor, securing the vehicle to the epidermal layer in the desired orientation, and removing the vehicle from the epidermal layer after a period of time, thereby inducing hair growth in the desired orientation.

21. The method of claim 20, wherein the mammal is a human.

22. The method of claim 20, wherein the stiff shaft is a tube.

23. The method of claim 20, wherein the stiff shaft is a rigid thread.

24. The method of claim 20, wherein the stiff shaft is a rod.

25. The method of claim 20, wherein the vehicle further comprises a growth factor.

26. The method of claim 20, wherein the period of time is between about two and about ten days.

27. The method of claim 20, wherein the vehicle further comprises an eccentrically expressed growth/transcription factor.

28. The method of claim 27, wherein the eccentrically expressed growth/transcription factor is member of the Wnt growth/transcription factor family.

29. The method of claim 27, wherein the eccentrically expressed growth/transcription factor is member of the sonic hedgehog growth/transcription factor family.

30. The method of claim 27, wherein the eccentrically expressed growth/transcription factor is a member of the Krox-20 growth/transcription factor family.

31. The method of claim 27, wherein the eccentrically expressed growth/transcription factor is a member of the homeobox growth/transcription factor family.

32. The method of claim 27, wherein the eccentrically expressed growth/transcription factor is a member of the Notch growth/transcription factor family.

33. The method of claim 27, wherein the eccentrically expressed growth/transcription factor is a member of the transforming growth factor-alpha growth/transcription factor family.

34. The method of claim 27, wherein the eccentrically expressed growth/transcription factor is a member of the insulin-like growth factor growth/transcription factor family.

35. An apparatus for the packaging and placement of papilla cells, the apparatus comprising at least one tube, wherein the tube comprises papilla cells that are sealed in individual segments of the tube, thereby forming an apparatus for the packaging and placement of papilla cells.

36. The apparatus of claim 35, wherein one or more tubes are interconnected by at least one flexible device.

37. The apparatus of claim 35, wherein the material of the tube comprises a biologically inert plastic.

38. The apparatus of claim 35, wherein the tube is sealed by means consisting of heat sealing, chemical sealing, and mechanical sealing.

39. The apparatus of claim 35, wherein the individual segments are detached from the tube to form a vehicle comprising a stiff shaft and at least one papilla cell.
